(19) United States
(12) Patent Application Publication
(10) Pub. No.: US 2020/0179222 A1
(43) Pub. Date: Jun. 11, 2020

(54) Massage tool with electrotherapy

(71) Applicant: Jeremy Quick, DeForest, WI (US)

(72) Inventor: Jeremy Quick, DeForest, WI (US)

(21) Appl. No.: 16/212,375

(22) Filed: Dec. 6, 2018

(51) Int. Cl. A61H 99/00; A61N 1/36; A61N 1/32

(52) U.S. Cl. CPC A61H 99/00; A61N 1/36003; A61H 2201/10; A61N 1/322; A61N 1/36014

(57) ABSTRACT

The present invention relates to the combination of both an instrument or tool meant for massage or instrument assisted soft tissue mobilization and electrotherapy. Current tools do not allow for simultaneous use of the two modalities. This tool is coated with an epoxy-based powder coat that is often used in electrical components. This provides insulation from the electricity that is being run through the tool to the patient. There is also a location for the insertion of a wire that will connect to the electrotherapy device. The use of the two together will allow for the practitioner to combine the benefits of both instrument assisted soft tissue mobilization and electrotherapy while saving time.

### FIELD OF INVENTION

The present invention relates to instrument assisted soft tissue mobilization (ISTM) and the use of therapeutic electrotherapy in a clinical setting. More specifically, the invention relates to the use of both modalities simultaneously to increase the effectiveness of treatment.

### BACKGROUND

Manual therapy has employed the use of both ISTM and electrotherapy for years. Traditionally, they have been used individually, with ISTM being used to increase range of motion. Electrotherapy has been used to manage pain, decrease inflammation, reduce disuse atrophy, etc.

While there may have been practitioners who utilize the two treatments within the same visit, there haven't been any to utilize an ISTM tool to administer electrotherapy. While both modalities have benefits that they offer to the patient, the potential opportunities increase once the two are being applied simultaneously.

Many drawbacks exist when using both modalities separately. The largest drawback being time. Practitioners have limited time to treat each individual patient, so utilizing both modalities will allow for more time to devote to rehabilitation exercises, stretches, etc. The traditional treatment time in electrotherapy is approx. 8 min-20 min. The traditional soft tissue treatment time is approx. 5 min-10 min. Utilizing the two together could potentially cut treatment times in half.

Another drawback to solely using ISTM, is pain during and after treatment. It is well documented that anything causing pain, is increasing the sensitivity to pain in the future. The pain associated from treatment is most likely from the notion of breaking of scar tissue to allow for more range of motion. This is in essence a trauma, with pain and inflammation being the by-product.

A drawback also exists in the use of electrotherapy for pain management. It is often debated whether or not the use of electrotherapy has any long-term benefits in the management of pain. With the lack of a clear, concise answer, one can assume that electrotherapy may or may not be a beneficial treatment in relation to pain.

Therefore, a need exists to not only to decrease the amount of time that a patient spends with a patient working with one modality, but in increasing the efficacy of both modalities in seeking positive results post-treatment.

The proposed tool using electrotherapy and ISTM simultaneously solves the aforementioned issues by combining two modalities that have been utilized in treatments issued by manual therapists for many years.

### BRIEF SUMMARY OF THE INVENTION

The present invention combines the two modalities in one stainless steel tool. The design of the tool is such that the practitioner is being protected from the tool by not utilizing all edges of the tool for treatment edges, as is the case with traditional tools. The tool is also powder coated with an epoxy-based powder that provides electrical insulation to the tool. Electrotherapy is introduced to the tool by two holes made in the corner of the tool. One hole is meant for the attachment site of a wire that connects to most electrotherapy devices, like that of an electrode. The adjacent hole is threaded for a set screw to hold the wire in place.

### BRIEF DESCRIPTION OF THE DRAWINGS

Some embodiments of the present invention are illustrated as an example and are not limited by the figures of the accompanying drawing.

The image FIG. 1 depicts a side profile of a tool used to combine electrotherapy and ISTM according to various embodiments described herein.

FIG. 2 is a perspective view depicting a tool that is an example of a tool used to combine electrotherapy and ISTM according to various embodiments described herein.

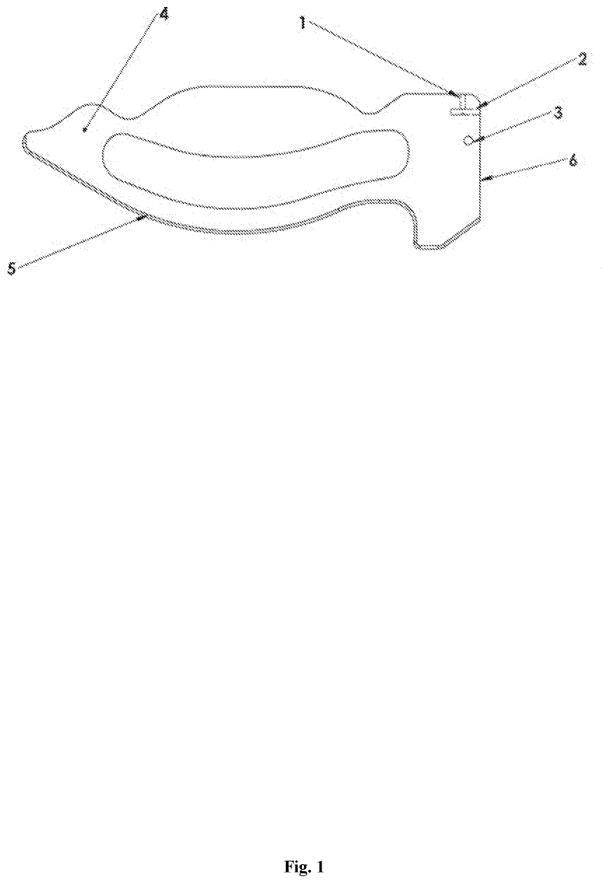

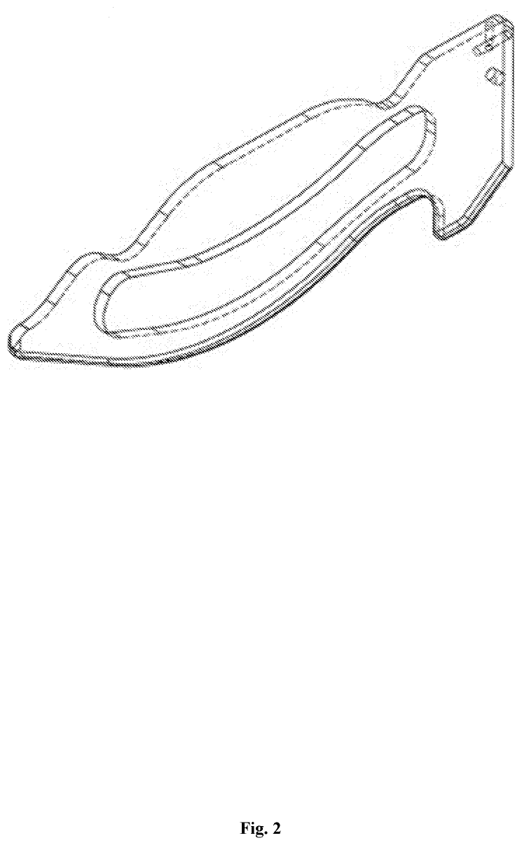

### DETAILED DESCRIPTION OF THE INVENTION

The terminology used herein is for the purpose of describing particular embodiments only and is not intended to be limiting of the invention. Unless otherwise defined, all terms (including technical and scientific) used herein have the same meaning as commonly understood by one having ordinary skill in the art to which this invention belongs.

In describing the invention, it will be understood that a number of ways exist to allow for the combination of these two modalities. This description represents the most current means to combine the two, and electro myofascial technique (ISTM combined with electrotherapy) is not limited to the invention described.

This device and method of application is designed to allow manual therapists, physicians, etc. to perform both instrument assisted soft tissue mobilization and electrotherapy simultaneously.

The device and method of application will now be described while making reference to the figures provided. The side view in FIG. 1 depicts the design of the tool itself to enable the user to comfortably and effectively hold the tool while treating the patient. It is noted by the number 4 that the tool is to be powder coated with an epoxy-based powder. This is used to exploit the die-electric strength of epoxy powder, which has been used in insulating electrical components for years.

Treatment edges/surfaces are depicted by the number 5, and these surfaces allow for the practitioner to use a variety of treatment edges during one treatment session. Edges marked with the number 6 are meant to be non-treatment edges, and will remain powder coated to insulate the practitioner from the electrical current.

In order to connect a wire that is typically found on an electrotherapy electrode, the hole that is to be machined denoted by the number 1 is a tapped hole that is to house a set screw that will hold the wire in place. The wire is to be inserted into the hole labeled by the number 2 in order to pass the electrical current from the electrotherapy device, through the tool, and ultimately be used to facilitate treatment of the patient. The final hole that is labeled 3, is a hole that is meant to be the location of a metal wire that will hang the part in order to prepare/wash the stainless steel for powder coating, as well as the powder coating process itself.

The tool is to be, but not limited to, the following process in order to include all aspects needed for the tool to be effective and safe. The design of tool is first cut out by a laser. It is then washed with an acetone-based wash while being hung on a rack. Once the steel has thoroughly dried, it remains on the rack while it is sprayed with an epoxy-based powder coat. The tool is then placed in an oven, at 400 degrees Fahrenheit, for 20 minutes. The tool is then machined on a 3 axis CNC machine to attain the treatment edge and the holes labeled 1 and 2 on the FIG. 1. Once the machining process is finished, the wire is attached and a set screw is tightened to ensure the wire remains inserted in the tool.

Application of the tool is described as follows, but is not limited to this description. An emollient or lotion is placed on the patient's skin to reduce friction during treatment. The tool is then used to locate fibrous adhesions within the musculature of the patient. Once located, the electrotherapy is connected to the tool with the positive lead connecting to the tool, and the negative lead attached proximally in relation to the treatment area. The electrotherapy is then turned on to the patient's comfort level, and the practitioner administers the ISTM treatment.

What is claimed is:

1. The combination of a massage tool, regardless of medium or design, combined with electrotherapy for use in treating musculoskeletal condition
